(19) United States
(12) Patent Application Publication
(10) Pub. No.: US 2023/0406888 A1
(43) Pub. Date: Dec. 21, 2023

(54) METHODS AND SYSTEMS FOR TARGETING AUTOIMMUNE AND INFLAMMATORY PATHWAYS USING NANOLIGOMERS

(71) Applicant: Sachi Bioworks Inc., Louisville, CO (US)

(72) Inventor: Prashant Nagpal, Lafayette, CO (US)

(73) Assignee: Sachi Bioworks Inc., Louisville, CO (US)

(21) Appl. No.: 18/137,101

(22) Filed: Apr. 20, 2023

Related U.S. Application Data
(60) Provisional application No. 63/409,294, filed on Sep. 23, 2022.
(60) Provisional application No. 63/335,485, filed on Apr. 27, 2022.
(60) Provisional application No. 63/344,152, filed on May 20, 2022.
(60) Provisional application No. 63/390,909, filed on Jul. 20, 2022.

(51) Int. Cl. C07K 14/00; B82Y 5/00; A61K 33/242; A61P 25/00

(52) U.S. Cl. CPC C07K 14/003; A61P 25/00; A61K 33/242; B82Y 5/00

(57) ABSTRACT

Compositions and methods for targeting autoimmune and inflammatory pathways using Nanoligomers are disclosed herein. Nanoligomers may include a sequence, such as a polynucleotide or a peptide nucleic acid, connected to a nanostructure, such as a nanoparticle. A nanoligomer may downregulate a component of the NLRP3 inflammasome, such as NLRP3. A nanoligomer may downregulate NF-κβ. Compositions comprising nanoligomers may be used to treat a variety of diseases associated with autoimmune disease and inflammation.

### CROSS-REFERENCE TO RELATED APPLICATIONS

This application claims the benefit of priority of U.S. Provisional Patent Application Ser. No. 63/409,294, filed on Sep. 23, 2022, and titled “METHODS AND SYSTEMS FOR TARGETING NEUROINFLAMMATORY PATHWAYS USING NANOLIGOMERS,” and U.S. Provisional Patent Application Ser. No. 63/335,485, filed on Apr. 27, 2022, and titled “METHODS AND SYSTEMS FOR REVERSING RADIATION-INDUCED IMMUNOSUPPRESSION,” and U.S. Provisional Patent Application Ser. No. 63/344,152, filed on May 20, 2022, and titled “OLIGOMER FOR MITIGATION OF NEUROINFLAMMATORY DISEASE, AND A METHOD FOR MANUFACTURING THE SAME,” and U.S. Provisional Patent Application Ser. No. 63/390,909, filed on Jul. 20, 2022 and titled “METHOD FOR MITIGATING A VIRAL AGENT INFECTION IN A SUBJECT AND SYSTEM FOR BIODISTRIBUTING A NANOLIGOMER IN A SUBJECT,” each of which is incorporated by reference herein in its entirety.

### FIELD OF THE INVENTION

The present invention generally relates to the field of health and biology. In particular, the present invention is directed to methods and systems for targeting autoimmune and inflammatory pathways using Nanoligomers™.

### BACKGROUND

Neuroinflammation is a key factor in the development of neurodegenerative diseases, including prion disease. Pathogenesis includes accumulation of misfolded proteins, synaptic dysfunction, and cognitive/behavioral deficits followed by irreversible neuronal death, with no effective treatments to halt or slow the progression.

Even beyond neuroinflammation, same pathways are implicated in a range of autoimmune diseases such as Ulcerative Colitis, Crohn's Disease, Psoriasis, and many others. Unintended activation of human immune system which attacks healthy cells and organs represents one of the biggest unmet clinical need and pharmaceutical market.

### SUMMARY OF THE DISCLOSURE

In an aspect, a nanoligomer includes a targeting sequence, wherein the targeting sequence includes a polynucleotide binding domain capable of hybridizing with a polynucleotide encoding a component of the NLRP3 inflammasome; and a nanostructure.

In another aspect, a nanoligomer includes: a targeting sequence, wherein the targeting sequence includes a polynucleotide binding domain capable of hybridizing with a polynucleotide encoding NF-κβ; and a nanostructure.

In another aspect, a composition includes a first nanoligomer and a second nanoligomer; wherein the first nanoligomer includes a targeting sequence, wherein the targeting sequence includes a polynucleotide binding domain capable of hybridizing with a polynucleotide encoding a component of the NLRP3 inflammasome; and a nanostructure; wherein the second nanoligomer includes: a targeting sequence, wherein the targeting sequence includes a polynucleotide binding domain capable of hybridizing with a polynucleotide encoding NF-κβ; and a nanostructure.

In another aspect, a method of treating a subject in need thereof includes administering to the subject a composition includes a first nanoligomer and a second nanoligomer; wherein the first nanoligomer includes: a targeting sequence, wherein the targeting sequence includes a polynucleotide binding domain capable of hybridizing with a polynucleotide encoding a component of the NLRP3 inflammasome; and a nanostructure; wherein the second nanoligomer includes: a targeting sequence, wherein the targeting sequence includes a polynucleotide binding domain capable of hybridizing with a polynucleotide encoding NF-κβ; and a nanostructure.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary schematic of an experimental set-up in accordance with aspects of the disclosure;

FIG. 2 shows exemplary cognition and behavioral deficits are protected by nanoligomers treated by intraperitoneal route in accordance with aspects of the disclosure;

FIG. 3 shows exemplary microglia and astrocytic inflammation are significantly reduced using nanoligomers in prion diseased mice in accordance with aspects of the disclosure;

FIG. 4 shows exemplary prion induced spongiotic change and neuronal loss that is significantly decreased with Nanoligomer SB_NI_112 treatment in accordance with aspects of the disclosure;

FIG. 5 is a block diagram of an exemplary embodiment of a machine-learning module that may perform one or more machine-learning processes in accordance with aspects of the invention;

FIG. 6 shows additional exemplary cognition and behavioral deficits are protected by nanoligomers treated by intraperitoneal route in accordance with aspects of the disclosure;

FIG. 7 shows exemplary microglia and astrocytic inflammation are significantly reduced using nanoligomers in prion diseased mice in accordance with aspects of the disclosure;

FIG. 8 shows exemplary prion induced spongiotic change and neuronal loss that is significantly decreased with Nanoligomer SB_NI_112 treatment in accordance with aspects of the disclosure; and

FIG. 9 shows exemplary prion induced spongiotic change and neuronal loss that is significantly decreased with Nanoligomer SB_NI_112 treatment in accordance with aspects of the disclosure;

FIG. 10 shows results of a study examining SB_NI_112 availability within different regions of the brain;

FIG. 11A-F shows results of an animal model of autoimmune disease with or without SB_NI_112;

FIG. 12A-B depicts flow cytometry results and an experiment design for determining Treg populations; and

FIG. 13 is a block diagram of a computing system that can be used to implement any one or more of the methodologies disclosed herein and any one or more portions thereof in accordance with aspects of the invention.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Incorporated herein by reference is a sequence listing XML file entitled, “1186-006USU1 sequence listing 1.xml,” created on Apr. 18, 2023, and including a size of 16.1 KB (16,557 bytes).

In some embodiments, a nanoligomer described herein is capable of regulating the NLRP3 inflammasome. The NLRP3 inflammasome is a component of the innate immune system that, among other functions, modifies production and secretion of proinflammatory cytokines such as IL-1B and IL-18. Activation of the NLRP3 inflammasome is associated with inflammatory diseases such as Alzheimer's disease. In some embodiments, a nanoligomer is capable of downregulating NLRP3 inflammasome activity. In some embodiments, a nanoligomer is capable of downregulating expression of a component of the NLRP3 inflammasome. In some embodiments, a nanoligomer is capable of downregulating NF-κβ activity. In some embodiments, a composition comprises a nanoligomer capable of downregulating NLRP3 inflammasome activity and a nanoligomer capable of downregulating NF-κβ activity. In some embodiments, a subject in need thereof is treated by administration of a composition comprising one or more nanoligomers.

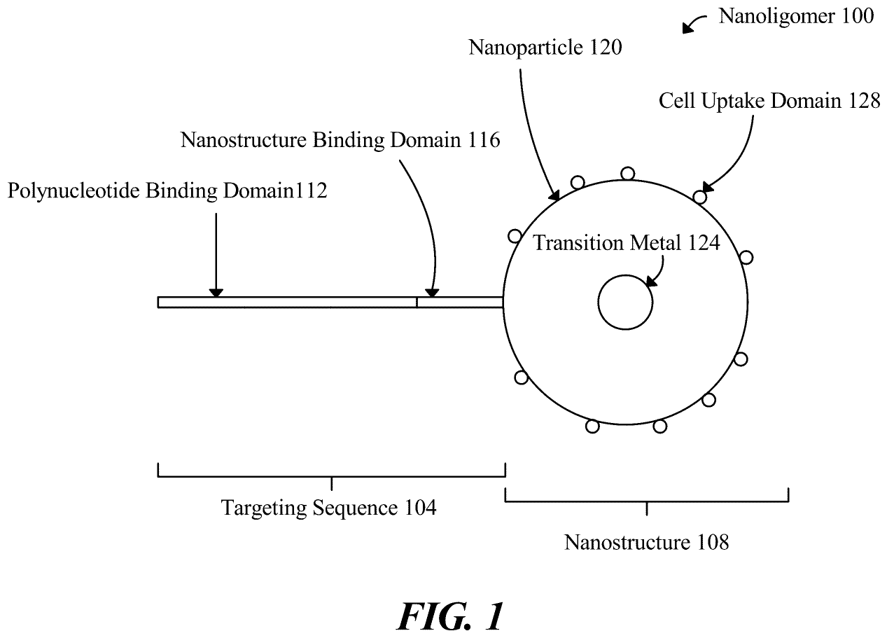

Referring now to FIG. 1, an exemplary embodiment of a system for targeting neuroinflammation is illustrated. A system may include a computing device. A system may include a processor. A processor may include, without limitation, any processor described in this disclosure. A processor may be included in a computing device. A computing device may any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a non-limiting example, using a “shared nothing” architecture.

Still referring to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, a nanoligomer 100 may include a targeting sequence 104 and a nanostructure 108. A targeting sequence 104 may include a polynucleotide binding domain 112 and a nanostructure binding domain 116. In some embodiments, a targeting sequence may further include a nuclear localization sequence. In some embodiments, a targeting sequence may further include a transcriptional activation domain. A nanostructure 108 may include a nanoparticle 120. A nanoparticle 120 may include a transition metal 124. A nanostructure 108 may include a cell uptake domain 128.

Still referring to FIG. 1, in some embodiments, a nanoligomer may include a nanoligomer disclosed in Table 1.

TABLE 1

| Description | Sequence |
|---|---|
| SB_NI_112 mouse | AGTGGTACCGTCTGCTA-AEEA-HHHHH-Au22, Glutathione 18 |
| NFKB1 | |
| SB_NI_112 mouse | CTTCTACTGCTCACAGG-AEEA-HHHHH-Au22, Glutathione 18 |
| NLRP3 | |
| SB_NI_112 human | CGGGTGCTTGCCATCTT-AEEA-HHHHH-Au22, Glutathione 18 |
| NLRP3 | |
| SB_NI_112 human | TGCCATTCTGAAGCCGG-AEEA-HHHHH-Au22, Glutathione 18 |
| NF-KB | |

Still referring to FIG. 1, in some embodiments, a nanoligomer 100 may include a targeting sequence 104. As used herein, a “targeting sequence” is a sequence of nucleobases including a polynucleotide binding domain and a nanostructure binding domain. A targeting sequence may include a peptide nucleic acid (PNA). As used in this disclosure, a “peptide nucleic acid” is a DNA analog comprising a (2-aminoethyl) glycine carbonyl unit, as opposed to a phosphate backbone, that is linked to a nucleotide base by the glycine amino nitrogen and/or methylene linker. In an embodiment, and without limitation, a PNA may include a backbone composed of peptide bonds linking nucleobases. In another embodiment, and without limitation, a PNA may include an amino-terminal and/or a carboxy-terminal end. In another embodiment, and without limitation, a PNA may include a 5′ and/or a 3′ end in the conventional sense, with reference to a complementary nucleic acid sequence to which it specifically hybridizes. In an embodiment, a PNA may include a sequence that may be described in a conventional fashion similar to DNA and/or RNA, such as but not limited to having nucleotides including guanine (G), uracil (U), thymine (T), adenine (A), and/or cytosine (C) which may correspond to a nucleotide sequence of a DNA molecule. In an embodiment, a PNA may be resistant to proteases and/or nucleases as a function of a structural difference from DNA, wherein the structure difference may result in a PNA not being recognized by a hepatic transporter(s) recognizing DNA. In another embodiment, a PNA may comprise at least one modified phosphate backbone such as, but not limited to phosphorothioate, phosphorodithioate, 5-phosphoramidothioate, phosphoramidate, phosphordiamidate, methylphosphonate, alkyl phosphotriester, formacetal, and/or the like thereof. In some embodiments, one or more of a polynucleotide binding domain, a nanoparticle binding domain, a nuclear localization sequence, and a transcriptional activation domain includes a PNA. A targeting sequence may include a polynucleotide, such as DNA or RNA. A targeting sequence may include an antisense oligonucleotide. As used in this disclosure, an “antisense oligonucleotide” is an antisense molecule that modulates the expression of one or more genes and/or polynucleotides. For example, and without limitation, an antisense oligonucleotide may include antisense PNAs, antisense RNAs, and the like. In another embodiment, antisense oligonucleotides may include RNA and/or DNA oligomers such as but not limited to interfering RNA molecules, such as dsRNA, dsDNA, mRNA, siRNA, and/or hpRNA as well as locked nucleic acids, BNA, polypeptides and/or other oligomers and the like thereof. A nanoligomer may include an inhibitory sequence. An “inhibitory sequence,” as used in this disclosure, is a sequence of nucleotides or other repeating units that acts to suppress production of neuroinflammatory elements such as proteins or the like. An inhibitory sequence may include, without limitation, sequences of ribonucleic acid (RNA), deoxyribonucleic acid (DNA), or peptide nucleic acid (PNA). In an embodiment, a targeting sequence is complementary to a sequence of interest. In an embodiment, an antisense oligonucleotide is complementary to a sequence of interest. In an embodiment, an inhibitory sequence is complementary to a sequence of interest. As used in this disclosure, a “complementary” sequence is a sequence of consecutive nucleobases or semi-consecutive nucleobases capable of hybridizing to another nucleic acid strand or duplex even if less than all the nucleobases base pair with a counterpart nucleobase. In some embodiments, between about 70% and about 100%, or any range derivable therein, of a nucleobase sequence may be capable of base-pairing with a nucleic acid molecule during hybridization.

Still referring to FIG. 1, a targeting sequence 104 may include a polynucleotide binding domain 112. As used herein, a “polynucleotide binding domain” is a sequence of nucleobases capable of hybridizing with a target polynucleotide. In some embodiments, a polynucleotide binding domain is complementary to a target polynucleotide. In some embodiments, a target polynucleotide encodes a proinflammatory cytokine, a direct inflammasome target, or a transcription factor. In some embodiments, a target polynucleotide encodes TERT, a cytokine selected from the list Interleukin-1β or IL-1β, IL-1α, tumor necrosis factor-alpha or TNF-α, TNF receptor 1 or TNFR1, Interleukin 6 or IL-6, IL-4, and IL-13. In some embodiments, a target polynucleotide encodes an inflammasome target selected from the list NLRP1, NLRP3, NLRC4, AIM2. In some embodiments, a target polynucleotide may encode NLRP3. In some embodiments, a target polynucleotide may encode NF-κβ. In some embodiments, a polynucleotide binding domain targeting mouse NFKB1 has the sequence AGTGGTACCGTCTGCTA. In some embodiments, a polynucleotide binding domain targeting mouse NLRP3 has the sequence CTTCTACTGCTCACAGG. In some embodiments, a polynucleotide binding domain targeting human NLRP3 has the sequence CGGGTGCTTGCCATCTT. In some embodiments, a polynucleotide binding domain targeting human NF-κβ has the sequence TGCCATTCTGAAGCCGG. In some embodiments, a target polynucleotide may include a sequence disclosed in Table 2. In some embodiments, a polynucleotide binding domain may be capable of hybridizing with a sequence disclosed in Table 2.

TABLE 2

| Description | Sequence |
|---|---|
| Human NLRP3 RNA | CGGGTGCTTGCCATCTT |
| Human NLRP3 RNA | GTGCTTGCCATCTTCAT |
| Human NLRP3 RNA | CTTGCCATCTTCATCTG |
| Human NLRP3 RNA | CCATCTTCATCTGCAGC |
| Human NLRP3 RNA | CAGCGGGTGCTTGCCAT |
| Human NF-κβ RNA | TTCTGCCATTCTGAAGC |
| Human NF-κβ RNA | TGCCATTCTGAAGCCGG |
| Human NF-κβ RNA | CCATTCTGAAGCCGGGT |
| Human NF-κβ RNA | ATCATCTTCTGCCATTC |
| Human NF-κβ RNA | ATCTTCTGCCATTCTGA |
| Mouse NFKB1 RNA | AGTGGTACCGTCTGCTA |
| Mouse NLRP3 RNA | CTTCTACTGCTCACAGG |

Still referring to FIG. 1, in some embodiments, a polynucleotide binding sequence may be capable of hybridizing with a section of a sequence in Table 2. In non-limiting examples, a polynucleotide binding sequence may be capable of hybridizing to a 10, 11, 12, 13, 14, 15, 16, or 17 nucleotide long stretch of a sequence in Table 2.

Still referring to FIG. 1, in some embodiments, a polynucleotide binding sequence may include a sequence selected from SEQ ID NO: 1-4. In some embodiments, a polynucleotide binding sequence may be capable of hybridizing with a sequence selected from SEQ ID NO: 5-16.

Still referring to FIG. 1, in some embodiments, a target polynucleotide may include RNA, such as mRNA. In some embodiments, hybridization of a polynucleotide binding sequence to a target mRNA may sterically hinder a ribosomal binding site and downregulate translation from the mRNA. In some embodiments, hybridization of a polynucleotide binding sequence to a target mRNA may prevent translation of the RNA. In some embodiments, a target polynucleotide may include DNA. In some embodiments, hybridization of a polynucleotide binding sequence to a target DNA may prevent transcription of the DNA.

Still referring to FIG. 1, a targeting sequence 104 may include a nanostructure binding domain 116. As used herein, a “nanostructure binding domain” is a nucleobase sequence containing a binding domain for a nanostructure. In some embodiments, a nanostructure binding domain may include a sequence of 5, 6, or 7 consecutive histidine (H in Table 1), 5, 6, or 7 consecutive cysteine, 5, 6, or 7 methionine, or 5, 6, or 7 lysine. In some embodiments, a nanostructure binding domain may include a sequence of 5 consecutive histidine. In some embodiments, a nanostructure binding domain may include SEQ ID NO: 18.

Still referring to FIG. 1, a targeting sequence 104 may include a linker. In some embodiments, a linker may include the sequence AEEA. In some embodiments, a linker may include SEQ ID NO: 17. In some embodiments, a spacer may be positioned between nanostructure binding domain and the rest of the targeting sequence.

Still referring to FIG. 1, a targeting sequence 104 may include a nuclear localization sequence. In some embodiments, a targeting sequence includes a polynucleotide binding sequence targeting DNA and a nuclear localization sequence. In some embodiments, a targeting sequence includes a polynucleotide binding sequence targeting RNA and no nuclear localization sequence.

Still referring to FIG. 1, in some embodiments, a targeting sequence 104 may include a transcriptional activation domain. In some embodiments, inclusion of a transcriptional activation domain may cause nanoligomer to upregulate transcription of a sequence of interest.

Still referring to FIG. 1, in some embodiments, a nanoligomer 100 may include a nanostructure 108. In some embodiments, a nanoligomer may include a targeting sequence bound to a nanostructure. As used in this disclosure a “nanostructure” is a structure of intermediate size between microscopic and molecular structures. For example, and without limitation, nanostructure may include a structure that comprises a size in the range of 0.1 nm to 100 nanometers. In an embodiment, and without limitation, a nanostructure 108 may include a nanoparticle 120. In an embodiment, and without limitation, a targeting sequence may be bound to a nanostructure via a covalent bond. As used in this disclosure a “covalent bond” is a chemical bond that involves sharing of electrons between atoms. For example, and without limitation, covalent bond may include electron pairs that are shared and/or bonded as a function of a stable balance of attractive and/or repulsive forces between atoms. In an embodiment, and without limitation, a covalent bond may allow molecules and/or atoms to fill one or more valence shells of an atom to produce a stable electronic configuration. In another embodiment, a covalent bond may include one or more interactions such as, but not limited to σ-bonding, π-bonding, metal-to-metal bonding, agnostic interactions, bent bonds, three-center two-electron bonds, three-center four-electron bonds, and the like.

Still referring to FIG. 1, a nanostructure 108 may include a nanoparticle 120. As used herein a “nanoparticle” is a three-dimensional object existing on a nanoscale, wherein the particle is between 0.1 nm and 100 nm in each spatial dimension. A nanoparticle may be spherical. For example, and without limitation, a nanoparticle may include a spherical nanoparticle with a diameter of about 23 nm.

Still referring to FIG. 1, in some embodiments, a nanoparticle 120 may include a transition metal. In some embodiments, a nanoparticle may include gold. In some embodiments, a nanoparticle may include Au 22. As used in this disclosure a “transition metal nanoparticle” is a nanoparticle composed of a transition metal. For example, and without limitation, a transition metal nanoparticle may include a gold nanoparticle. As a further non-limiting example, a transition metal nanoparticle may include a copper nanoparticle. As a further non-limiting example, a transition metal nanoparticle may include a zinc nanoparticle. In an embodiment, and without limitation, a transition metal nanoparticle may include one or more transition metals comprising groups 3-12 transition metals on the period table of elements.

Still referring to FIG. 1, in some embodiments, a nanostructure 108 may include a cell uptake domain 128. In some embodiments, a cell uptake domain may include glutathione. In some embodiments, a cell uptake domain may include glutathione 18.

Still referring to FIG. 1, in some embodiments, a composition, such as a therapeutic composition, may include a nanoligomer. In some embodiments, a composition may include more than one type of nanoligomer. In some embodiments, a composition may include a first nanoligomer including a targeting sequence targeting NLRP3 and a second nanoligomer including a targeting sequence targeting NF-κβ.

Still referring to FIG. 1, in some embodiments, a nanoligomer 100 may be capable of regulating the expression of a target gene. In some embodiments, a nanoligomer may be capable of upregulating transcription of a target gene. In some embodiments, a nanoligomer may be capable of downregulating transcription of a target gene. In some embodiments, a nanoligomer may be capable of downregulating expression of a gene by regulating mRNA translation. In some embodiments, a nanoligomer may be capable of downregulating expression of a gene by decreasing RNA stability.

Still referring to FIG. 1, in some embodiments, administration of a nanoligomer 100 to a subject may have a therapeutic or prophylactic effect. As used herein, a “therapeutic effect” is a reduction or elimination of a symptom of a disease in a subject. In some embodiments, administration of a nanoligomer to a subject may result in a therapeutic or prophylactic effect in the central nervous system. In some embodiments, administration of a nanoligomer to a subject may result in a therapeutic or prophylactic effect in the brain. In some embodiments, a nanoligomer is capable of crossing the blood brain barrier.

Still referring to FIG. 1, in some embodiments, a nanoligomer 100 may be capable of regulating the NLRP3 inflammasome. In some embodiments, a nanoligomer may be capable of downregulating expression of the NLRP3 gene. In some embodiments, a nanoligomer may include a targeting sequence capable of hybridizing with a polynucleotide encoding NLRP3. In some embodiments, a nanoligomer may include a targeting sequence capable of hybridizing with mRNA encoding NLRP3. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NLRP3. In some embodiments, a nanoligomer may be capable of downregulating expression of the NF-κβ gene. In some embodiments, a nanoligomer may include a targeting sequence capable of hybridizing with a polynucleotide encoding NF-κβ. In some embodiments, a nanoligomer may include a targeting sequence capable of hybridizing with mRNA encoding NF-κβ. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NF-κβ. In some embodiments, a nanoligomer may include a targeting sequence capable of hybridizing with a polynucleotide encoding a gene selected from IL-1β, IL-1α, TNF-α, IL-6, IL-4, IL-13, AIM2, TNRF1, NLRP1, NLRP6, NLRC4, NLRP3, and NF-κβ.

Still referring to FIG. 1, in some embodiments, a composition comprising a nanoligomer targeting NLRP3 and a nanoligomer targeting NF-κβ is administered to a subject. In some embodiments, administration of a NLRP3 nanoligomer and a NF-κβ nanoligomer may lead to a reduction in IL-18 levels compared to a nanoligomer targeting only one of them or compared to a control. In some embodiments, administration of a NLRP3 nanoligomer and a NF-κβ nanoligomer may lead to a reduction in IL-1 levels compared to a nanoligomer targeting only one of them or compared to a control. In some embodiments, administration of a NLRP3 nanoligomer and a NF-κβ nanoligomer may lead to a reduction in IL-4 levels compared to a nanoligomer targeting only one of them or compared to a control. In some embodiments, administration of a NLRP3 nanoligomer and a NF-κβ nanoligomer may lead to a reduction in CD30 levels compared to a nanoligomer targeting only one of them or compared to a control. In some embodiments, administration of a NLRP3 nanoligomer and a NF-κβ nanoligomer may lead to a reduction in IL-31 levels compared to a nanoligomer targeting only one of them or compared to a control. In some embodiments, administration of a NLRP3 nanoligomer and a NF-κβ nanoligomer may lead to a reduction in CXCL5 levels compared to a nanoligomer targeting only one of them or compared to a control. In some embodiments, administration of a NLRP3 nanoligomer and a NF-κβ nanoligomer may lead to a reduction in CCL4 levels compared to a nanoligomer targeting only one of them or compared to a control. In some embodiments, administration of a NLRP3 nanoligomer and a NF-κβ nanoligomer may lead to a reduction in CCL20 levels compared to a nanoligomer targeting only one of them or compared to a control. In some embodiments, administration of a NLRP3 nanoligomer and a NF-κβ nanoligomer may lead to a reduction in CXCL11 levels compared to a nanoligomer targeting only one of them or compared to a control. In some embodiments, administration of a NLRP3 nanoligomer and a NF-κβ nanoligomer may lead to a reduction in CD40L levels compared to a nanoligomer targeting only one of them or compared to a control.

Still referring to FIG. 1, in some embodiments, one or more nanoligomers are formulated with a pharmaceutically acceptable excipient. As used herein, a “pharmaceutically acceptable excipient” is a pharmaceutically acceptable material, composition or vehicle involved in carrying or transporting a payload from one cell-type, organ, or portion of the body to another cell-type, organ, or portion of the body. Pharmaceutically acceptable excipients include, for example, solvents, dispersion media, coatings, antibacterial and antifungal agents, isotonic and absorption delaying agents, liquid or solid fillers, diluents, excipients, manufacturing aids (such as lubricants, talc magnesium, calcium or zinc stearate, or steric acid), or solvent encapsulating materials. Each pharmaceutically acceptable excipient may be “acceptable” in the sense of being compatible with the other ingredients of the formulation and not injurious to the subject. Some examples of materials which may serve as pharmaceutically-acceptable excipients include, without limitation: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (18) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants.

Still referring to FIG. 1, a composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

Still referring to FIG. 1, a composition described herein may be administered to a subject that has a disease, or that has a risk of developing a disease. As an example, a composition described herein may be administered to a subject that has a disease associated with neuroinflammation. A composition described herein may be administered to a subject that has a disease selected from the list IBD, Alzheimer's disease, Parkinson's disease, multiple sclerosis, prion's disease/Creutzfeldt-Jakob disease (CJD), neurodegenerative diseases, autoimmune diseases, cancer, liver fibrosis, NASH, diabetes, gout, myocardial infarction, and sepsis. In some embodiments, a disease may be treated via a method including administering to a subject one or more nanoligomers, or a composition including one or more nanoligomers. For example, a disease may be treated by administering a composition comprising a nanoligomer targeting NF-κβ DNA or mRNA, and a nanoligomer targeting NLRP3 DNA or mRNA. As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Still referring to FIG. 1, in some embodiments, a composition described herein may be administered to a subject that has inflammatory bowel disease (IBD). In some embodiments, a composition described herein may be administered to a subject that has a risk of developing IBD. In some embodiments, IBD may refer to Chron's disease and ulcerative colitis. In some embodiments, IBD may be characterized by chronic inflammation of the GI tract. In some embodiments, a composition described herein may be administered to a subject that has Chron's disease. In some embodiments, a composition described herein may be administered to a subject that has ulcerative colitis.

Still referring to FIG. 1, in some embodiments, a therapeutically effective amount of a composition, such as a composition including one or more nanoligomers, is administered to a subject. As used herein, an “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” will vary depending on the composition, the condition and its severity, the manner of administration, and the age of the subject to be treated. As used herein, a “subject” is a mammal that has a disease or condition of interest, or that has a risk of developing a disease or condition of interest. In some embodiments, a subject is a Homo sapiens.

Neuroprotection using an immunotherapeutic nanoligomer in prion diseased mice could open new avenues for other neurodegenerative diseases such as but not limited to Parkinson's Disease, Prion Disease, Multiple Sclerosis, and Alzheimer's disease.

We hypothesized that downregulation of key immunotherapy targets: combination of NLRP3 inflammasome and transcription factor NF-κB, would be neuroprotective. To test this, we utilized Nanoligomers™ in a prion-diseased mouse to assess the impact on glial inflammation, behavioral/cognition deficits, aggregation of misfolded proteins, neuroinflammatory signaling, and loss of neurons. The treatment showed decreased numbers of microglia and S100β positive astrocytes, markers of neuroinflammation, and improved hippocampal behaviors and cognitive tests, indicating slowed disease pathogenesis. Critically, the Nanoligomer™ protected the brain from prion-induced spongiotic change, neuronal loss, and significantly increased life span of the mice showing that Nanoligomer™ inhibited key inflammatory pathways can prevent neuronal death and slow the progression of neurodegenerative diseases.

Neurogenerative diseases are on the rise impacting millions of people worldwide. These diseases are commonly characterized by an increase in glial inflammation or activation and aggregation of misfolded proteins that increase over time, followed by irreversible neuronal loss. Importantly there are no effective therapies that halt this disease progression allowing for neuronal protection. There are many laboratory models commonly utilized for the studying of these diseases, however few rodent models have all aspects of human neurodegenerative protein-misfolding diseases (NPMDs). Most transgenic mouse models for Alzheimer's disease (AD), AD related diseases (ADRDs) or Parkinson's diseases display few of the neuronal pathogenesis, behavioral and cognitive deficits or clinical sign phenotypes. To study therapeutic interventions, we used a wild-type mouse that actually develops a NPMD disease following inoculation with prions. Prion diseases are rare neurodegenerative diseases that undergo the common characteristics of NPMDs as the disease progresses. Previous literature has shown that therapeutic developed for prion diseased mice can be translated to other NPMDs (cite). While a few compounds have been shown to reduce signs of prion disease in mouse models, these compounds have toxic effects in the brain or elsewhere.

Prion diseases result from the native conformation of the cellular prion protein (PrPC) misfolding to the infectious form, PrPSc. This misfolding occurs when the alpha helical PrPC protein is conformationally changed to the β-sheet rich PrPSc, which forms amyloid fibrils and aggregates, causing the disruption of brain homeostasis. Similar to other NPMDs an early sign of prion disease, thought to be caused by the aggregation of PrPSc, is toxic glial inflammation with activation of microglia and astrocytes and systemic inflammation. However, it may be that PrPSc itself is not neurotoxic, and other cellular stress pathways, including glial inflammation, play a major role in disease pathogenesis. Neuroinflammation results in increased oxidative stress, secretion of cytokines, disruption of neural signaling, and glial scarring leading to the subsequent damage and loss of synapses, neuronal dysfunction and neuronal death. Chronic neuroinflammation is known to be damaging to nervous tissue and contributes to the development of prion diseases and other NPMDs.

To identify the optimal targets for tackling neuroinflammation cascade, we screened the downregulation of several proinflammatory cytokines (e.g., Interleukin-1β or IL-1β, tumor necrosis factor-alpha or TNF-α, Interleukin 6 or IL-6), inflammasomes (e.g., NLRP3, NLRP1), key transcription factors (e.g., nuclear factor kappa-B or NF-κβ), and their combinations. Using administration of non-toxic, targeted, effective, and bioavailable Nanoligomers that can be administered as naked molecules through injection or inhalation and designed to cross the blood-brain-barrier, we identified a combination of NLRP3 inflammasome and NF-κβ transcription factor as a viable candidate. During brain stress, like NPMDs, there is significant cross-talk between microglia and astrocytes which involves critical inflammatory cell signaling events, like NF-κβ and the inflammasome formation. The transcription factor, NF-κβ, when translocated into the nucleus causes a number of inflammatory cytokines and chemokines to be transcribed causing a cascade of microglia and astrocyte inflammation. Another cell signaling pathway shown to be critical for NPMDs, like prion diseases, is the activation of the inflammasome, specifically caused by the NLRP3 protein. We hypothesized that if we can inhibit both of these pathways simultaneously that prion disease and other neurodegenerative diseases such as Parkinson's Disease, Prion Disease, Multiple Sclerosis, and Alzheimer's disease, progression will slow, as a proof of principle for other NPMDs. Critically in this study, these Nanoligomers were able to save neurons, reduce spongiotic change, decrease behavioral and cognitive deficits and glial inflammation in prion diseased brains once the infection had already been ensued.

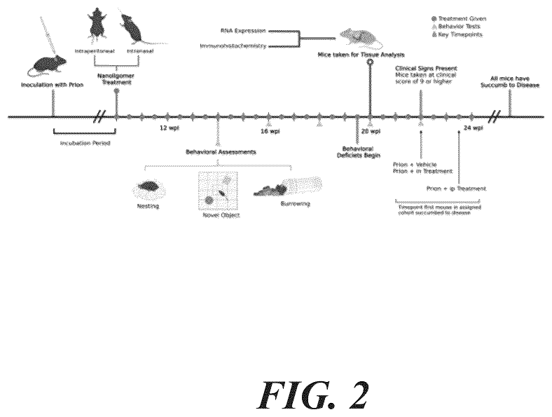

Protection of the Nanoligomer™ against prion-induced cognitive and behavioral deficits. Wild-type, C57Bl6/J, (Jackson laboratories) mice were infected intracerebrally with brain homogenates from Rocky Mountain Laboratories (RML) prions or normal brain homogenates (NBH) as the negative control. Nanoligomer treatment began at 10 weeks post inoculation (wpi) or approximately 40% of full disease progression and continued until the mice succumbed to prion disease. 10 mg/Kg of the Nanoligomer SB_NI_112 or saline as the vehicle, was given either intraperitoneally (i.p.) or intranasally (i.n.) twice a week to both prion and NBH mice, as shown in FIG. 1. As the disease progressed and treatment continued behavioral assessments including novel object recognition, burrowing, and nesting, were performed at 12 wpi to train and establish a baseline for each experimental cohort, shown in FIG. 2. All three behavioral assessments measure hippocampal integrity, the brain region known to show the first clinical pathology including glial inflammation and spongiotic change.

Mice with RML induced prion disease have a significant decrease in the ability to recognize a familiar object as this is prion strain that begins to aggregate within the hippocampus the brain region essential for memory and learning. Both i.p. and i.n. SB_NI_112 treatment protected prion induced deficit in novel object recognition when compared to vehicle treated diseased mice at 22 wpi, shown in FIG. 2B. The ratio of time the mouse spent with the novel object to the familiar object, seen 24 hours beforehand was calculated and noted the discrimination index. The closer the discrimination index is to one the more intact the hippocampal memory is. Both intraperitoneal treated mice (p<0.0005, mean discrimination index score: 0.449) and intranasal treated mice (p<0.005, mean discrimination index score: 0.434) had a significantly increase in novel object recognition, when compared to vehicle treated diseased mice (discrimination index of −0.174, ie. preferring the familiar object). At 20 wpi a significant rise in the mice to recognize the novel was also seen between vehicle and SB_NI_112 i.p. by analysis of the discrimination index, shown in FIG. 2A.

Two other common hippocampal specific behavioral assessments were performed on the cohorts of mice to compare prion induced deficits with and without SB_NI_112 treatment. Mice were given a plastic tube filled with food pellets within in their home cages, for 30 min to burrow freely. Following this time, the food pellets left in the tubes were weighed and percent burrowed calculated. Prion diseased mice are known to stop burrowing as disease progresses but prion diseased mouse treated with SB_NI_112 at 20 wpi, 21 wpi, and 22 wpi, did not decrease burrowing as statistically significant as vehicle only mice, shown in FIG. 2D, showing a trend of protection (detailed quantitative assessment scoring provided in Methods). Mice were given napkins to nest overnight and scored between one (no nest formed) and five (fluffy built nest) weekly starting at 16 wpi. The weights of the mice with and without treatment with SB_NI_112 were not significantly different throughout the disease progression indicating no acute toxicity due to the Nanoliogmer. Monitoring of nest formation was found to be significantly higher among i.p. treated mice at 20 wpi (p>0.005), 21 wpi (p>0.005), and 22 wpi (p>0.05), shown in FIG. 2C, compared to vehicle treated prion infected nest formation.

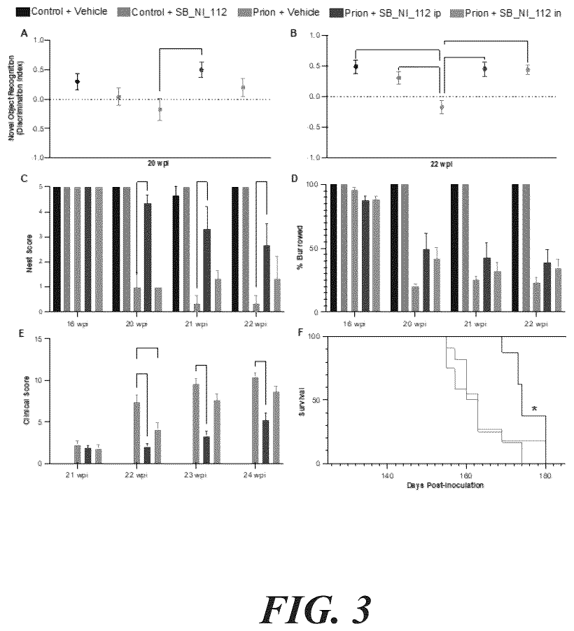

Nanoligomer™ SB_NI_112 treatment reduces glial inflammation. In addition to the behavioral assessments to quantify hippocampal learning deficits during disease progression, potential therapeutic effect was assessed using immunohistology and biochemical characterization. Brain tissues were collected at 20 wpi and 24 wpi from all treatment groups. Formalin fixed tissues were processed and embedded into paraffin wax. Once brains were sliced on the microtome brains were stained for both microglial and astrocytic inflammation, shown in FIG. 3. Astrocytic inflammation was identified as S10013+ cells, shown in FIG. 3A, and Iba1+ cells denote inflamed microglia, shown in FIG. 3F. Four brain regions known to be implemented in RML prion infection are the hippocampus, cortex, thalamus and cerebellum. In the thalamic region, both astrocytic and microglial inflammation was significantly suppressed with SB_NI_112 Nanoligomer treatment, shown in FIG. 3C and FIG. 3H, respectively, at 20 wpi. Additionally, both i.n. and i.p. SB_NI_112 treatment provided significant protection from microglia inflammation in the hippocampus, shown in FIG. 3G, cortex, shown in FIG. 3I, and the cerebellum, shown in FIG. 3J.

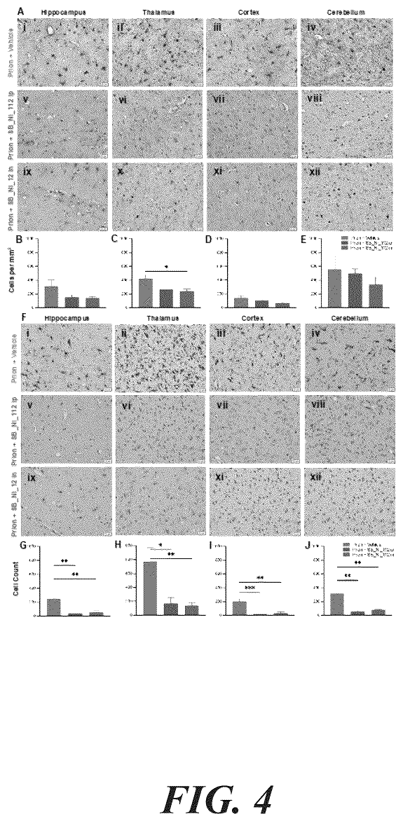

Neuronal protection using Nanoligomer™ SB_NI_112. A morphologic spongiotic or vacuoles within the brain tissue is a hallmark of prion disease that increases as the disease progresses. To assess and quantify this change, we used pathologic scoring of the hippocampus, thalamus, cortex, and cerebellum following H&E staining. This revealed protection against spongiotic change in both i.p. and i.n. SB_NI_112 Nanoligomer treatment of prion diseased mice, shown in FIGS. 4A and B, when compared to vehicle treated prion diseased mice. The loss of neurons within the hippocampus following RML prion infection is well established therefore we assessed this within our treatment group. Critically, neuronal numbers within the CA1 region of the hippocampus were significantly protected by SB_NI_112 Nanoligomer i.p. and i.n. treatment compared to vehicle treated (p>0.05), show in FIGS. 4C and 4D. Importantly, we see no change in the PK resistant prion protein PrPsc with Nanoligomer™ treatment (FIG. 4C). Therefore, the neuroprotection is independent of aggregation and misfolding of PrP^{Sc} itself and could be a good avenue of therapy for other NPMDs with differing toxic misfolded proteins.

Clinical scores and survival improved in mice treated with Nanoligomer. At 22 wpi, vehicle treated mice begin to show a significant increase clinical signs of prion disease, shown in FIG. 2E. From 22 wpi to 24 wpi i.p. and i.n. SB_NI_112 treated diseased mice have significantly (p<0.0001 and p<0.0005 respectively) lower clinical scores than vehicle treated diseased mice. Prion clinical signs included tail rigidity, hyperactivity, ataxia, extensor reflex, tremors, righting reflex, kyphosis, and poor grooming. Each sign was rated on a scale from 0-5. All clinical sign scores were combined for a total score. Mice were considered terminal and euthanized after reaching a total score of 9 or above. Date of mice succumbing to prion disease was documented for survival analysis, shown in FIG. 2E. Importantly, the lifespan of prion disease mice was significantly increased with i.p. SB_NI_112 Nanoligomer treatment (p>0.05) compared to vehicle treated mice.

The slowing of the progression of neurodegenerative protein misfolding diseases (NPMDs) has been proven to be difficult. Many promising treatments are toxic or must be delivered directly to the brain making translation to human patients difficult. Here we have successfully used a non-toxic, systemically delivered, Nanoligomer that protects against prion disease and other neurodegenerative disease, such as Parkinson's Disease, Prion Disease, Multiple Sclerosis, and Alzheimer's disease, phenotypes. Protection of cognition and other hippocampal behavioral deficits and the clinical progression of disease is accomplished with the Nanoligomer, as shown in FIG. 2. This Nanoligomer is specifically inhibiting two known inflammatory events, NF-κB and NLRP3 expression, in the brain during these diseases and significantly decreases microglial and astrocytic inflammation, slowing disease progression for prion disease and other neurodegenerative disease, such as Parkinson's Disease, Prion Disease, Multiple Sclerosis, and Alzheimer's disease. For instance, it may be slowing disease progression through the inhibition of glial inflammation, neurons were protected shown by a decrease in spongiotic change and neuronal loss, independent of a decrease in the misfolded protein itself. Critically, these findings show that these Nanoligomers can slow both early cognitive/behavioral phenotypic changes and later clinical signs, neuronal loss and death. Critically, we have identified a treatment for a neurodegenerative disease that is non-toxic and can be delivered to the periphery. These findings of neuroprotection within a prion diseased mouse can be easily translated to other neurodegenerative disease as neuroinflammation is a generic pathology irrespective of the misfolded protein.

Referring now to FIG. 2, an exemplary schematic of experimental set-up is shown.

Still referring to FIG. 2, prion diseased mice were treated with Nanoligomers or vehicle through intraperitoneal or intranasal routes of exposure, twice a week starting at 10 weeks post inoculation (10 wpi). Throughout the progression of the disease cognitive, hippocampal specific behaviors and clinical signs were monitored. At 20 wpi brain tissue was dissected for analysis of disease progression including neuroinflammation, spongiotic change and neuronal loss.

Referring now to FIG. 3, an exemplary embodiment of cognition and behavioral deficits that are protected by Nanoligomers treated by intraperitoneal route.

Still referring to FIG. 3, mice were monitored for changes in cognition and behavior throughout the disease progression. Nanoligomer treatment (SB_NI_112) protected the prion induced deficit in spatial novel object recognition seen in the vehicle treated prion diseased mice when given intraperitoneal (i.p.) for 20 wpi, as shown in A, and i.p and intranasal (i.n) at 24 wpid, as shown in B. As shown in C, no significant change was seen in the hippocampal assay burrowing when mice were treated with the nanoligomers compared to vehicle. Further, as shown in D, a significant increase in the ability of the mice to build proper nests at 20 and 21 wpi with i.p. treatment of the nanoligomers compared to vehicle treatments. As shown in E, prion clinical scores were monitored and shown to be protected by both intranasal and i.p. treatment at 22 wpi and continued to protective with i.p. until 25 wpi. Additionally, as shown in F, the life-span of these prion diseased mice was significantly elongated with i.p. nanoligomer treatment. N=9 for prion positive i.p. and i.n. groups, N=12 for all other groups. One-way ANOVA and post-hoc Tukey test, error bars=SEM, * p<0.05, ** p<0.01, *** p<0.001, **** p<0.0001, ns=not significant.

Referring now to FIG. 4, an exemplary embodiment of microglia and astrocytic inflammation that are significantly reduced using Nanoligomers in prion diseased mice.

Still referring to FIG. 4, Section A shows exemplary representative images of the hippocampus (i, v, ix), thalamus (ii, vi, x), cortex (iii, vii, xi) and cerebellum (iv, viii, xii) brain regions of S100b+ cells, a maker of inflamed astrocytes, with SB_NI_112 intraperitoneal (i.p.) and intranasal (i.n.) treatment. Quantitative analysis of each brain region identifies a significant change in the thalamus, shown in graph C, and a trend of a decrease of inflamed astrocytes in the hippocampus, shown in graph B, and cortex, as shown in D, and cerebellum, as shown in E, with treatment. Section F shows exemplary representative images of the hippocampus (i, v, ix), thalamus (ii, vi, x), cortex (iii, vii, xi) and cerebellum (iv, viii, xii) brain regions for IBA1+ cells, a maker for microglia, with and without treatment. Quantitative analysis of each brain region identifies a significant change in the hippocampus, as shown in G, thalamus, as shown in H, cortex, as shown in I, and cerebellum, as shown in J, with both i.n. and i.p. treatment. Arrows represent examples of positive cells. Scale bar=20 μm N=3-4. One-way ANOVA *p>0.05, **p>0.001, ***p>0.0001

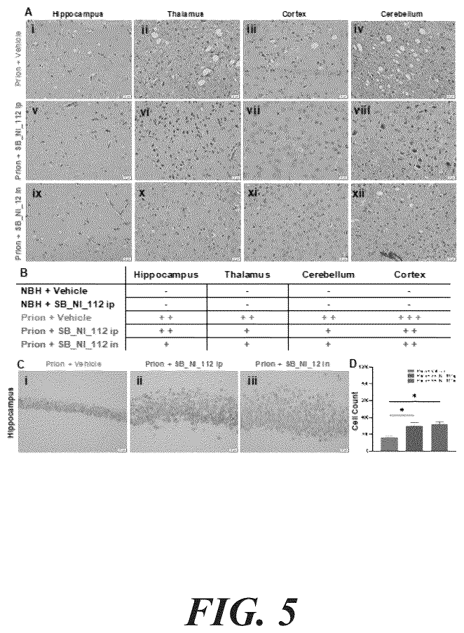

Referring now to FIG. 5, an exemplary embodiment of Prion induced spongiotic change and neuronal loss is significantly decreased with Nanoligomer SB_NI_112 treatment is shown.

Still referring to FIG. 5, Section A shows Representative images of the spongiotic change in hippocampus (i, v, ix), thalamus (ii, vi, x), cortex (iii, vii, xi) and cerebellum (iv, viii, xii) brain regions were protected with SB_NI_112 intraperitoneal (i.p.) and intranasal (i.n.) treatment. Section B shows exemplary Pathological scoring of each brain region identifies a protection or decrease of pathological score at all brain regions analyzed. Arrows point to spongiotic change. Section C shows exemplary representative images of the NeuN+ cells (neuronal cell bodies) in the CA1 region of the hippocampus. Section D shows exemplary significant decrease in the number of neurons lost at 20 wpi with treatment via i.p. and i.n. SB_NI_112. Scale bar=20 mm N=3-4. One-way ANOVA *p>0.05.

Materials and Methods

Ethics Approval

Mice were euthanized by deeply anaesthetizing with isoflurane followed by decapitation. All mice were bred and maintained at Lab Animal Resources, accredited by the Association for Assessment and Accreditation of Lab Animal Care International, in accordance with protocols approved by the Institutional Animal Care and Use Committee at Colorado State University.

Nanoligomer Development

Nanoligomers were synthesized using previously published procedure. Briefly, the Sachi bioinformatics platform was used to identify the lead Nanoligomer sequence of the DNA/RNA binding domain (DBD/RBD). The desired peptide molecules were synthesized using solid phase peptide synthesis, and conjugated to the gold nanoparticle, for high-throughput purification and delivery. The concentration of the peptide molecules and Nanoligomers was quantified using ultraviolet-visible (UV-Vis) optical spectroscopy.

Mice and Brain Homogenates

C57Bl/6 (Jackson Laboratory) mice were intracranially inoculated with 30 μl of 1% 22 L or Rocky Mountain Laboratories (RML) strains of mouse-adapted prions, or normal brain homogenate (NBH). Mice were monitored for weight loss and clinical signs of prion disease and euthanized after showing signs of terminal illness. 20% brain homogenates in phosphate-buffered saline (PBS) were made using beads and a tissue homogenizer (Benchmark Bead Blaster 24) and stored at −80 C. Brain homogenates were aliquoted and treated with UV light for 30 minutes to sterilize before being used for cell culture.

Nanoligomer Administration. At 10 weeks post RML prion or NBH inoculation, mice were treated two times per week either intraperitoneally (i.p.) or intranasally (i.n.) with 10 mg/kg of Nanoligomer SB_NI_112 diluted in sterile saline. Mice receiving treatment intranasally were anesthetized prior to administration and laid in an intranasal apparatus that controlled isoflurane throughout the intranasal procedure.

Cognitive Assay: Novel Object Recognition

Mice were tested in a rectangular arena (28 cm×43 cm). Mice were habituated to the arena seven days before testing. On day one of habituation, mice were permitted to explore the empty arena for ten minutes. On day two of habituation, two identical objects (constructed with Legos) were placed in the arena, approximately 10 cm from the walls with 20 cm distance between. Each mouse was placed in the arena for ten minutes of exploration. During day one of testing, mice were placed in the arena with two identical objects and were given 5 minutes of exploration. On day two of testing, one of the known objects was replaced by a novel object, and the 5-minute exploration was filmed using a 1080P FHD Mini Video Camera. All objects and the arena were cleansed thoroughly between trials to ensure the absence of olfactory cues. Time exploring both the known and novel object was calculated blinded. Discrimination index calculated following Absolute vs Relative Analysis protocol.

Hippocampal Behavioral Assays: Burrowing and Nesting

Briefly, mice were placed in a large cage with a PVC tube full of food pellets, as described (23). The natural tendency of rodents is to displace (burrow) the food pellets. The percentage of burrowing activity is calculated from the difference in the weight of pellets in the tube before and after 2 hours. To test nesting we used three fresh napkins were placed in each cage. After 24 hours, cages were examined for nesting activity. Nests were scored on a scale of 0-5, where 0 represents no nesting activity, and 5 represents a high-quality nest.

Clinical Scoring of Mice

Eight key clinical signs were monitored in mice daily beginning at 20 wpi. Clinical signs included tail rigidity, hyperactivity, ataxia, extensor reflex, tremors, righting reflex, kyphosis, and poor grooming. Each sign was rated on a scale from 0-5. All clinical sign scores were combined for a total score. Mice considered terminal and euthanized after reaching a total score of 9 or above.

Immunohistochemistry

Paraffin-embedded brains were sectioned at 4 um and stained with NeuN antibody (1:250; Cell Signaling) for CA1 neuronal counts. Astrogliosis was detected with Iba1 antibody (1:400; Abcam). S100B antibody (AbCam; 1:400) was used for microglial counts. Nonspecific binding was blocked before primary antibodies with 10% Horse Serum (Vector Labs). A biotinylated secondary antibody (Vector Labs) was used, and stain was visualized with diaminobenzidine reagent. All images were taken with *** software and counted with CellSens (Iba1 and S100B) or manually (NeuN).

Reverse Transcriptase Quantitative PCR Analysis

RNA was extracted from cell culture 6-well dishes using cell scraping, QIAshredder and RNeasy extraction kits, in accordance with manufacturer's protocol, including a DNase digestion step with the RNase free DNase kit (Qiagen, Valencia, CA). Purity and concentration were determined using a ND-1000 spectrophotometer (NanoDrop Technologies, Wilmington, DE). Following isolation and purification, 25 ng of RNA was reverse transcribed using the iScript Reverse Transcriptase kit (BioRad, Hercules CA). The cDNA was amplified within 24 hours of reverse transcription using iQ SYBR Green Supermix (BioRad, Hercules CA). The corresponding validated primer sequences were used for each gene at 10□M. The expression data was analyzed using the 2-ΔΔCT method and normalized to expression of reference genes β-actin. The fold difference was compared to control (normal brain homogenate treated) samples (27). Validated primer sequences are as follows:

(β-actin)
5′-CCACTGTCGAGTCGCGT-3′ (forward),
5′-CGCAGCGATATCGTCATCCAT-3′ (reverse);
(NLRP3)
5′-CCTGGGGGACTTTGGAATCA-3′ (forward),
5′-GACAACACGCGGATGTGAGA-3′ (reverse);
(IL1β)
5′-GCAGCAGCACATCAACAAG-3′ (forward),
5′-CACGGGAAAGACACAGGTAG-3′ (reverse);
(NF-κB1)
5′-GTGGAGGCATGTTCGGTAGT-3′ (forward),
5′-CCTGCGTTGGATTTCGTGAC-3′ (reverse);
(TNFR1a)
5′-GTTGTCAATTGCTGCCCTGTC-3′ (forward),
5′-CAGTGACCCCTGATGGATGT-3′ (reverse).

All RT-PCR was done following MIQE guidelines.

Immunoblotting

Brain homogenates were isolated using phosphate buffered saline (PBS) supplemented with Phos-STOP and Complete protease inhibitors (Roche). A BCA Protein Assay kit (Thermo Scientific) was used to quantify protein concentration of lysates, and 25 μg of protein was digested with 20 μg/mL proteinase K (PK) (Roche) for PrPSc blots for 1 hour at 37 C. Digestion was terminated with 2 mM PMSF. For PrPC blots, 10 μg of samples was used. Samples were run using 4-20% acrylamide SDS page gels (BioRad) and then transferred onto PVDF blotting paper (MilliPore). Primary antibody Bar-224 (Cayman Chemical) was used at 1:5,000 in 5% non-fat milk in tris buffered saline with tween and HRP-conjugated secondary antibodies at 1:5,000 (Vector Laboratories). The protein antibody complex was visualized using SuperSignal West Pico PLUS Chemiluminescent Substrate (Thermo Scientific) and visualized with the BioRad ChemiDoc MP.

In some embodiments, AI or machine learning may be used to select one or more target genes. In some embodiments, AI or machine learning may be used to select one or more nanoligomers.

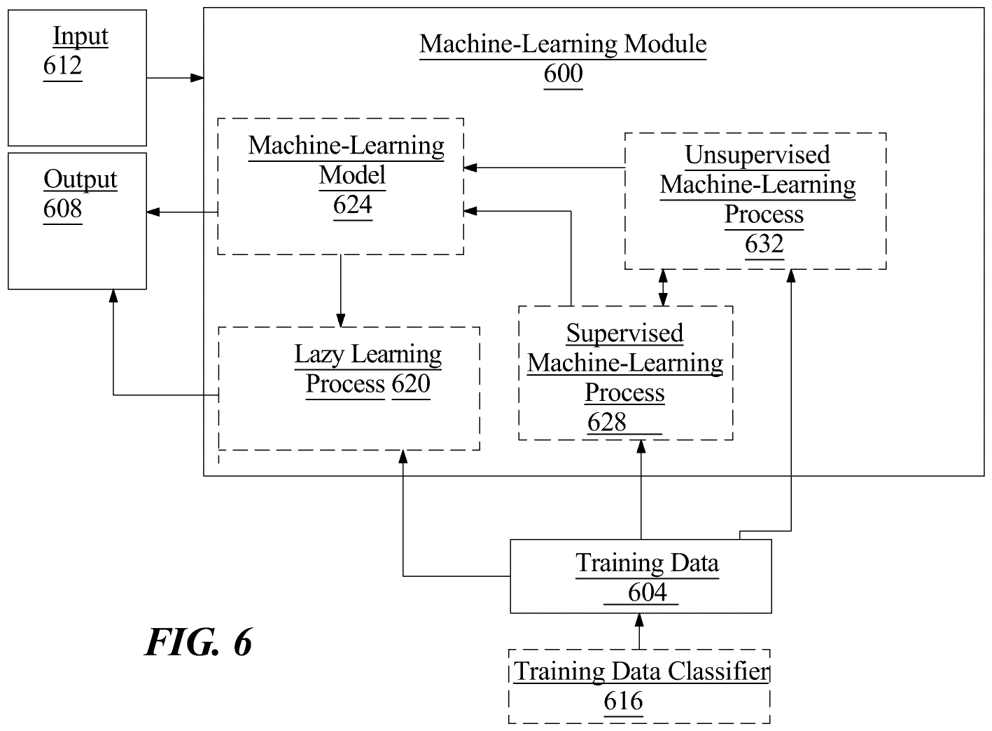

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to utilize potential match/mismatch of each candidate along the human genome.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described above as inputs, outputs as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

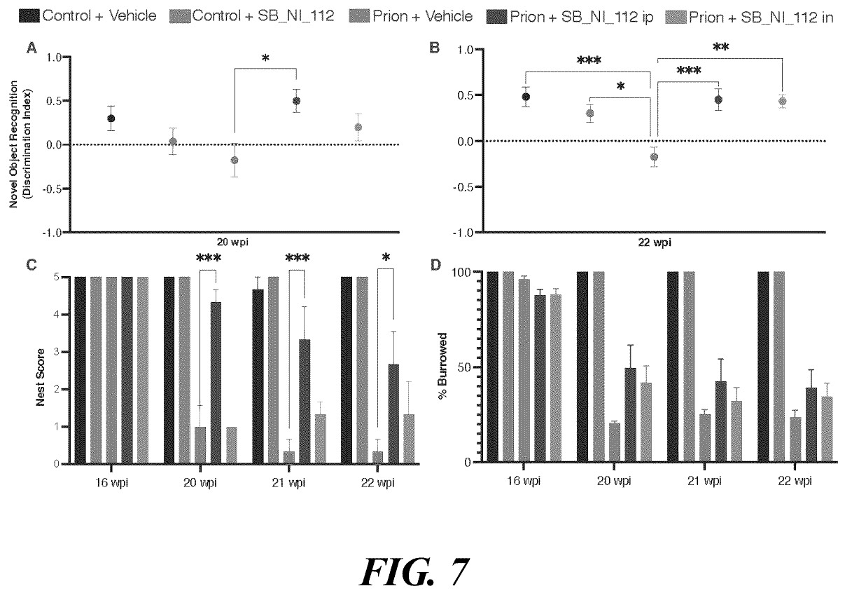

Now referring to FIG. 7, results of a prion disease mouse study are depicted. Wild type C57Bl6/J (prion disease model) mice were administered control+vehicle, control+SB_NI_112, prion+vehicle, prion+SB_NI_112 (intraperitoneal), or prion+SB_NI_112 (intranasal). Normal brain homogenates was used as the negative control. A and B: the ability of mice to recognize a familiar object was measured based on the ratio of time a mouse spent with a novel object to a familiar object seen 24 hours beforehand. A: intraperitoneal, 20 weeks post inoculation (wpi). B: intraperitoneal and intranasal, 24 wpi. C: Starting at 16 wpi, mice were given napkins to nest overnight and scored between 1 (no nest formed) and 5 (fluffy nest). D: Burrowing was also monitored: mice were placed in a cage with a plastic tube containing food pellets. After 30 min, the percent of pellets still in the tube was weighed. Error bars=SEM, * p<0.05, ** p<0.01, *** p<0.001, **** p<0.0001, ns=not significant.

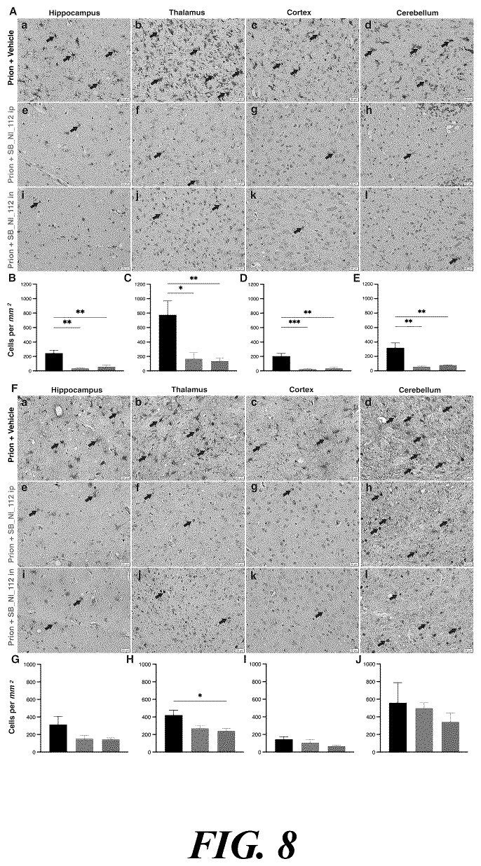

Now referring to FIG. 8, results of another prion disease mouse study are depicted. A: representative images of hippocampus, thalamus, cortex and cerebellum brain regions of S100β+ cells, a maker of inflamed astrocytes, with SB_NI_112 intraperitoneal and intranasal treatment is depicted. Cells per mm\hat{ }2 in the hippocampus (B), thalamus (C), cortex (D), and cerebellum (E) were measured. F: representative images of hippocampus, thalamus, cortex and cerebellum brain regions of IBA1+ cells, a maker for microglia with and without treatment was measured. Cells per mm\hat{ }2 in the hippocampus (G), thalamus (H), cortex (I), and cerebellum (J) were measured. *p>0.05, **p>0.001, ***p>0.0001

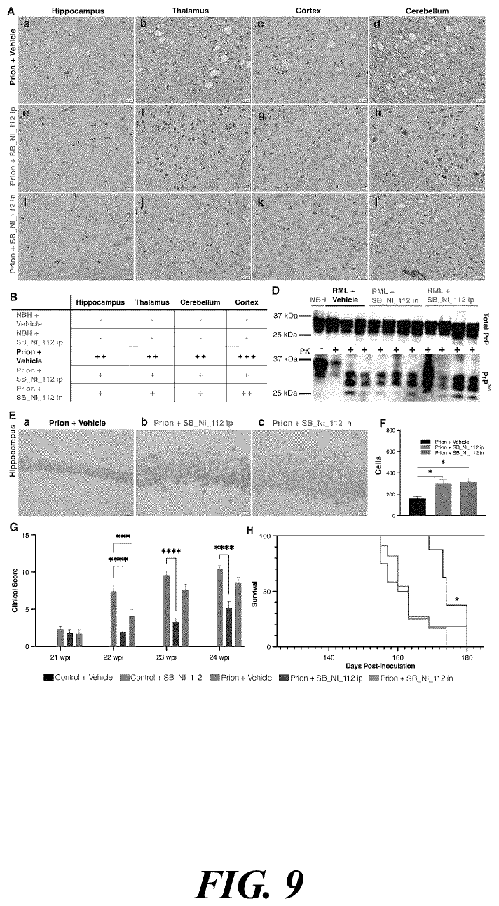

Now referring to FIG. 9, results of another prion disease mouse study are depicted. A: representative images of hippocampus, thalamus, cortex and cerebellum brain regions of mice given intraperitoneal and intranasal SB_NI_112 treatment is depicted. B: pathological scoring of each brain region. D: Representative images of the NeuN+ cells (neuronal cell bodies) in the CA1 region of the hippocampus. E: Neurons lost at 20 wpi with i.p. and i.n. SB_NI_112 treatment was measured. F: Prion clinical scores at 22 wpi with i.p. and i.n. SB_NI_112 treatment was measured. H: Life span of prion disease mice with varying treatments.

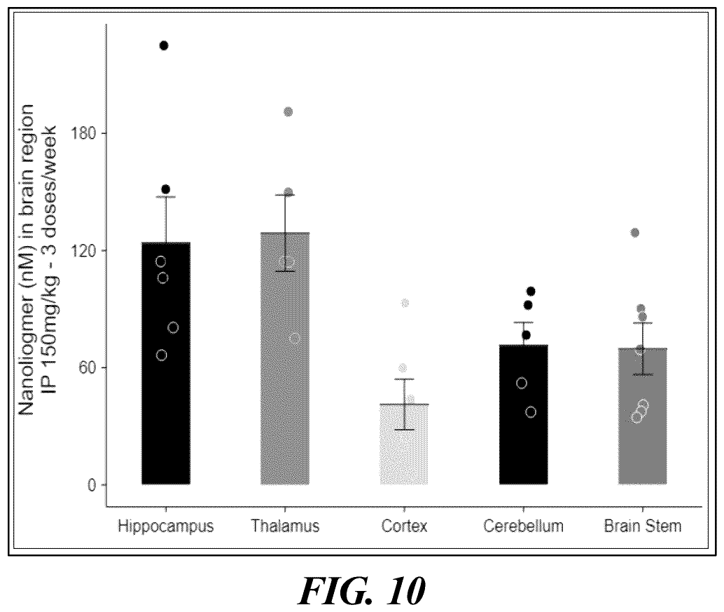

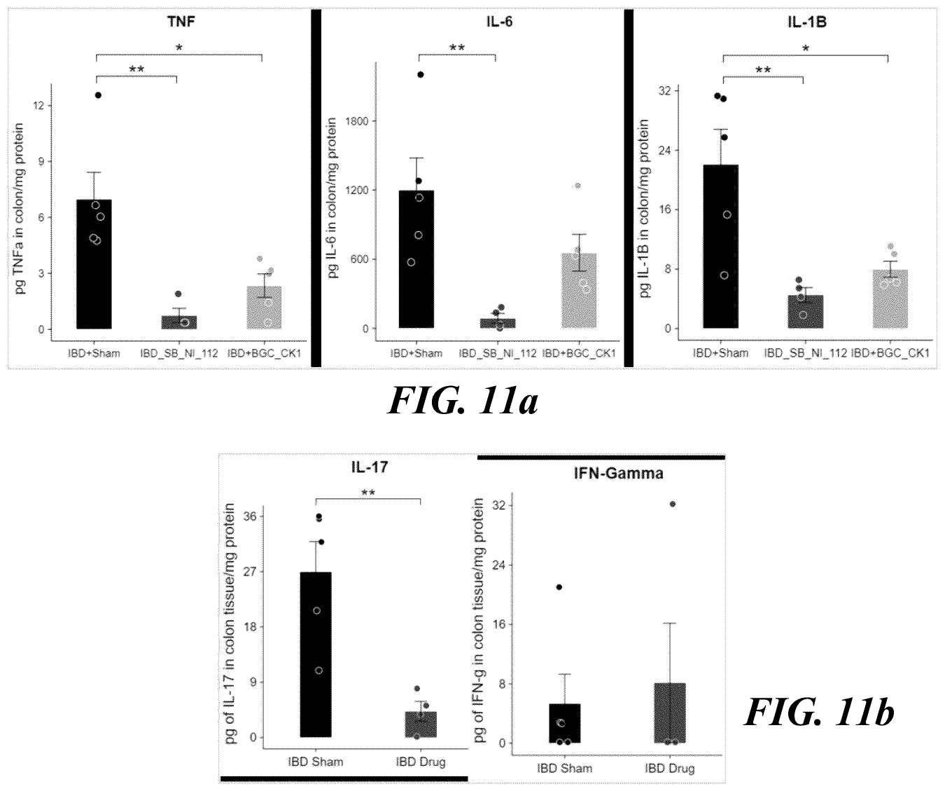

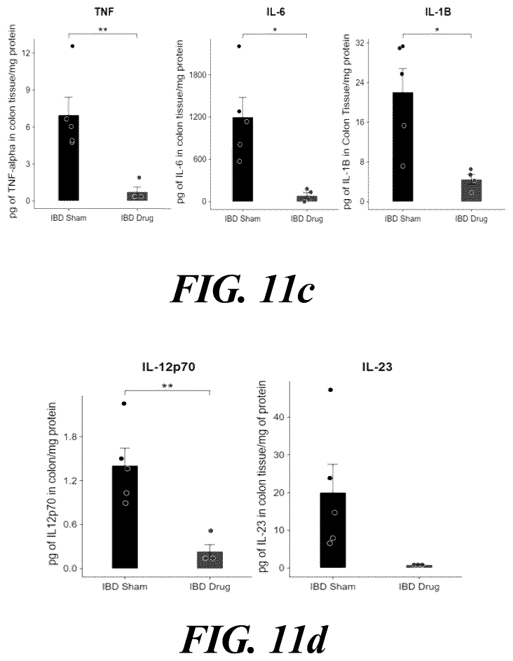

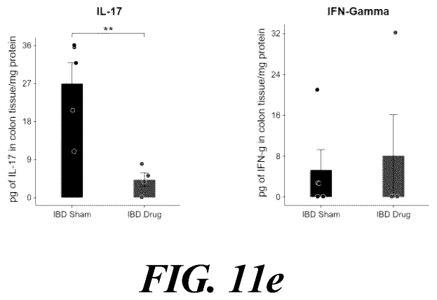

Now referring to FIG. 10, results of a study examining SB_NI_112 availability within different regions of the brain. Mice were dosed with IP administration of 150 mg/kg (maintenance dose) 3-times per week for 2 weeks. Mice were euthanized, brains were dissected using surgery to segregate different brain regions. Following organ collection, the tissues (with the Nanoligomer) were dissolved in Aqua Regia, which was then evaporated. The remaining solid was then re-suspended in nitric acid. Nanoligomer amounts were determined using Inductively Coupled Plasma Mass Spectrometry (ICP-MS).

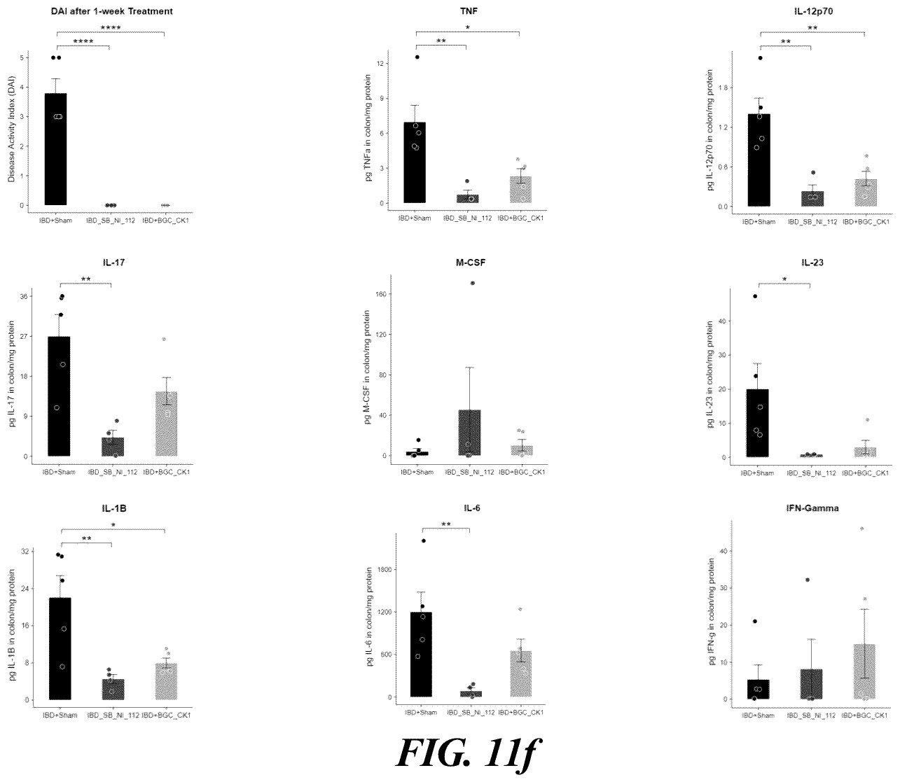

Now referring to FIG. 11, results of a study examining the role of SB_NI_112 in treating autoimmune disease such as Inflammatory Bowel Disease (IBD). Using an established Dextran Sodium Sulfate (DSS) Colitis model in mice, first C57BL-6 mice were acclimatized, their existing microbiome was wiped using 5-days of antibiotic administration, followed by recolonization with fecal gavage mixture from 10 IBD patients, to colonize mice gut with IBD-inducing microbiome. Following this, 3% DSS water was used to trigger the colitis in mice, and they were treated with either sham (saline solution) IP injection or empty (filler capsules), or treated with SB_NI_112 using IP injections or oral capsules. Following 3-dose treatment in one week, the mice colon were harvested, and analyzed for inflammation using histology and multiplexed ELISA.

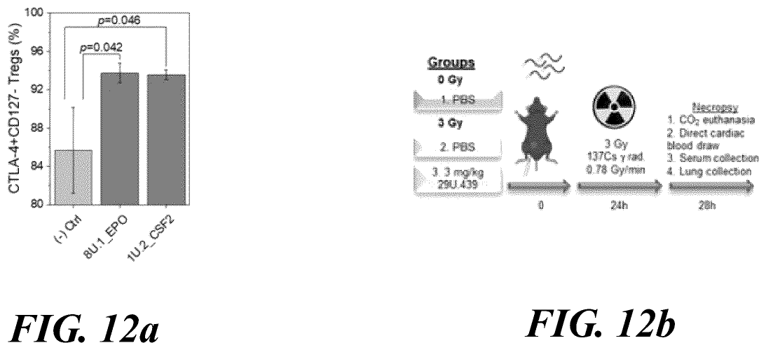

Now referring to FIG. 12, in some embodiments, a composition described herein may be administered to a subject that has an autoimmune disease or is at risk of developing an autoimmune disease. In some embodiments, a nanoligomer may regulate CSF2. While approved radioprotection therapies include Sargramostim, the impact of the inflammatory cytokine CSF2 recombinant protein on inducing further immune dysfunction especially on autoimmune diseases, including rheumatoid arthritis and multiple sclerosis has been implicated. Its potential role in astrocytosis and microgliosis leading to neurodegenerative diseases has also been revealed, leading to its use as a countermeasure target to treat radiation-induced neuropathy. However, the proposed gene-targeting to upregulate csf2 and epo resulted in upregulation of anti-inflammatory cytokines (for example IL-10) and other regulatory chemokines, pointing to controlled reversal of immune dysfunction, rather than overall increase of inflammatory markers. This also highlights the difference in immune response with recombinant protein (eg. Sargramostim) and gene therapy targeting protein upregulation.

Still referring to FIG. 12, in some embodiments, regulatory T (Treg) cells are essential for maintaining peripheral tolerance, preventing autoimmunity, and limiting chronic inflammatory diseases. To enumerate Treg cells, PBMCs were collected and stained for flow cytometry (FIG. 12B). Here we consider Tregs of relevance as the proportion of T cells that are CD25+ (Interleukin-2 receptor alpha chain) FoxP3+ (FoxP3: forkhead box P3, also known as scurfin) and their subpopulation that is CD127− (Interleukin-7 receptor alpha chain) CTLA4+ (cytotoxic T-lymphocyte-associated protein 4). To probe the impact of lead targets on Treg cell population, we conducted population-level immune regulation analysis with csf2 and epo upregulating Nanoligomers. To enumerate Treg cells, PBMCs were treated with 8U.1_EPO or 1U.2_CSF2 for 24 hours and then collected and stained for flow cytometry (FIG. 12A). We demonstrate that treatment with either 8U.1_EPO or 1U.2_CSF2 causes significant increase in Treg population compared to negative control (PBS). This observed Treg population increase highlights an additional difference in recombinant protein vs reversible gene therapy, and the potential role in regulating radiation dysfunction in multiple protein expression pathways using gene regulation networks.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

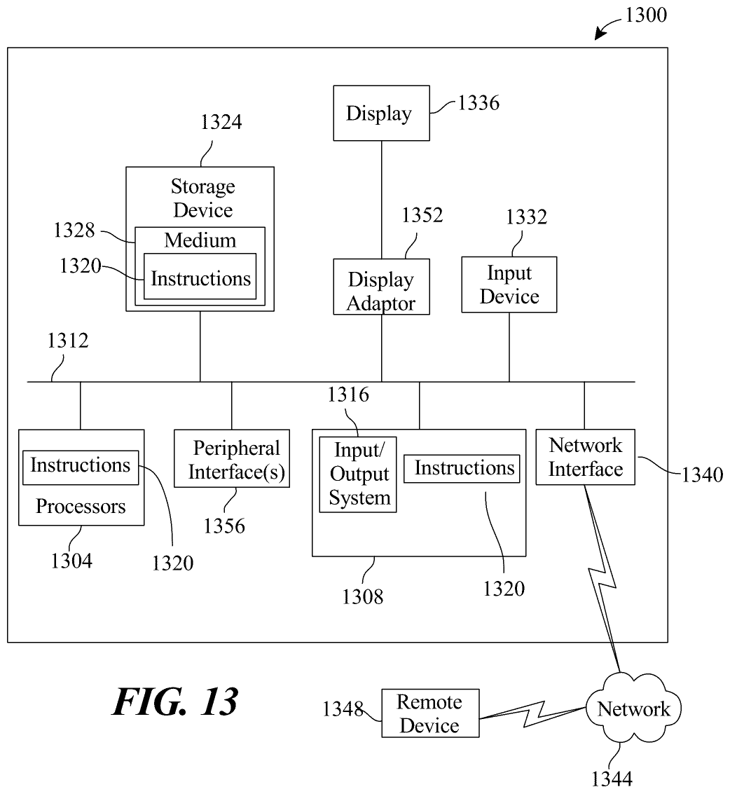

FIG. 13 shows a diagrammatic representation of one embodiment of a computing device in the exemplary form of a computer system 1300 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1300 includes a processor 1304 and a memory 1308 that communicate with each other, and with other components, via a bus 1312. Bus 1312 may include any of several types of bus structures including, but not limited to, a memory bus, a memory controller, a peripheral bus, a local bus, and any combinations thereof, using any of a variety of bus architectures.

Processor 1304 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1304 may be organized according to Von Neumann and/or Harvard architecture as a non-limiting example. Processor 1304 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1308 may include various components (e.g., machine-readable media) including, but not limited to, a random-access memory component, a read only component, and any combinations thereof. In one example, a basic input/output system 1316 (BIOS), including basic routines that help to transfer information between elements within computer system 1300, such as during start-up, may be stored in memory 1308. Memory 1308 may also include (e.g., stored on one or more machine-readable media) instructions (e.g., software) 1320 embodying any one or more of the aspects and/or methodologies of the present disclosure. In another example, memory 1308 may further include any number of program modules including, but not limited to, an operating system, one or more application programs, other program modules, program data, and any combinations thereof.

Computer system 1300 may also include a storage device 1324. Examples of a storage device (e.g., storage device 1324) include, but are not limited to, a hard disk drive, a magnetic disk drive, an optical disc drive in combination with an optical medium, a solid-state memory device, and any combinations thereof. Storage device 1324 may be connected to bus 1312 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1324 (or one or more components thereof) may be removably interfaced with computer system 1300 (e.g., via an external port connector (not shown)). Particularly, storage device 1324 and an associated machine-readable medium 1328 may provide nonvolatile and/or volatile storage of machine-readable instructions, data structures, program modules, and/or other data for computer system 1300. In one example, software 1320 may reside, completely or partially, within machine-readable medium 1328. In another example, software 1020 may reside, completely or partially, within processor 1304.

Computer system 1300 may also include an input device 1332. In one example, a user of computer system 1300 may enter commands and/or other information into computer system 1300 via input device 1332. Examples of an input device 1332 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1332 may be interfaced to bus 1312 via any of a variety of interfaces (not shown) including, but not limited to, a serial interface, a parallel interface, a game port, a USB interface, a FIREWIRE interface, a direct interface to bus 1312, and any combinations thereof. Input device 1332 may include a touch screen interface that may be a part of or separate from display 1336, discussed further below. Input device 1332 may be utilized as a user selection device for selecting one or more graphical representations in a graphical interface as described above.

A user may also input commands and/or other information to computer system 1300 via storage device 1324 (e.g., a removable disk drive, a flash drive, etc.) and/or network interface device 1340. A network interface device, such as network interface device 1340, may be utilized for connecting computer system 1300 to one or more of a variety of networks, such as network 1344, and one or more remote devices 1348 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1344, may employ a wired and/or a wireless mode of communication. In general, any network topology may be used. Information (e.g., data, software 1320, etc.) may be communicated to and/or from computer system 1300 via network interface device 1340.

Computer system 1300 may further include a video display adapter 1352 for communicating a displayable image to a display device, such as display device 1336. Examples of a display device include, but are not limited to, a liquid crystal display (LCD), a cathode ray tube (CRT), a plasma display, a light emitting diode (LED) display, and any combinations thereof. Display adapter 1352 and display device 1336 may be utilized in combination with processor 1304 to provide graphical representations of aspects of the present disclosure. In addition to a display device, computer system 1300 may include one or more other peripheral output devices including, but not limited to, an audio speaker, a printer, and any combinations thereof. Such peripheral output devices may be connected to bus 1312 via a peripheral interface 1356. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A Nanoligomer comprising:
a targeting sequence, wherein the targeting sequence comprises a polynucleotide binding domain capable of hybridizing with a polynucleotide encoding a component of the NLRP3 inflammasome; and
a nanostructure.

2. The Nanoligomer of claim 1, wherein the polynucleotide binding domain is capable of hybridizing with a polynucleotide encoding NLRP3.

3. The nanoligomer of claim 2, wherein the polynucleotide binding domain is capable of hybridizing with a sequence selected from the list consisting of SEQ ID NO: 5-9.

4. The Nanoligomer of claim 2, wherein the polynucleotide binding domain comprises SEQ ID NO: 3.

5. The Nanoligomer of claim 1, wherein the polynucleotide binding domain is a peptide nucleic acid.

6. The Nanoligomer of claim 1, wherein the targeting sequence comprises a nanostructure binding domain.

7. The Nanoligomer of claim 5, wherein the nanostructure binding domain comprises the sequence HHHHH.

8. The Nanoligomer of claim 1, wherein the targeting sequence comprises a linker.

9. The Nanoligomer of claim 8, wherein the linker comprises the sequence AEEA.

10. The Nanoligomer of claim 1, wherein the nanostructure comprises a transition metal nanoparticle.

11. The Nanoligomer of claim 10, wherein the transition metal nanoparticle comprises an Au 22 nanoparticle.

12. The Nanoligomer of claim 1, wherein the nanostructure comprises a cell uptake domain.

13. The Nanoligomer of claim 12, wherein the cell uptake domain comprises glutathione 18.

14. A Nanoligomer comprising:
a targeting sequence, wherein the targeting sequence comprises a polynucleotide binding domain capable of hybridizing with a polynucleotide encoding NF-κβ; and
a nanostructure.

15. The Nanoligomer of claim 2, wherein the polynucleotide binding domain is capable of hybridizing with a sequence selected from the list consisting of SEQ ID NO: 10-14.

16. The Nanoligomer of claim 15, wherein the polynucleotide binding domain comprises SEQ ID NO: 4.

17. The Nanoligomer of claim 15, wherein the polynucleotide binding domain is a peptide nucleic acid.

18. The Nanoligomer of claim 15, wherein the targeting sequence comprises a nanostructure binding domain.

19. The Nanoligomer of claim 18, wherein the nanostructure binding domain comprises SEQ ID NO: 18.

20. The Nanoligomer of claim 15, wherein the targeting sequence comprises a linker.

21. The Nanoligomer of claim 20, wherein the linker comprises SEQ ID NO: 17.

22. The Nanoligomer of claim 15, wherein the nanostructure comprises a transition metal nanoparticle.

23. The Nanoligomer of claim 21, wherein the transition metal nanoparticle comprises an Au 22 nanoparticle.

24. The Nanoligomer of claim 15, wherein the nanostructure comprises a cell uptake domain.

25. The Nanoligomer of claim 24, wherein the cell uptake domain comprises glutathione 18.

26. A composition comprising a first Nanoligomer and a second nanoligomer;
wherein the first Nanoligomer comprises:
a targeting sequence, wherein the targeting sequence comprises a polynucleotide binding domain capable of hybridizing with a polynucleotide encoding a component of the NLRP3 inflammasome; and
a nanostructure;
wherein the second Nanoligomer comprises:
a targeting sequence, wherein the targeting sequence comprises a polynucleotide binding domain capable of hybridizing with a polynucleotide encoding NF-κβ; and
a nanostructure.

27. The composition of claim 26, wherein the composition is formulated for oral administration.

28. A method of treating a subject in need thereof, comprising administering to the subject a composition comprising a first nanoligomer and a second Nanoligomer;
wherein the first Nanoligomer comprises:
a targeting sequence, wherein the targeting sequence comprises a polynucleotide binding domain capable of hybridizing with a polynucleotide encoding a component of the NLRP3 inflammasome; and
a nanostructure;
wherein the second Nanoligomer comprises:
a targeting sequence, wherein the targeting sequence comprises a polynucleotide binding domain capable of hybridizing with a polynucleotide encoding NF-κβ; and
a nanostructure.

29. The method of claim 28, wherein the composition is administered to the subject orally.


SEQUENCE LISTING (18 sequences)

SEQ ID NO: 1 - DNA, 17 bp, synthetic construct
  source 1..17: mol_type = other DNA
agtggtaccgtctgcta

SEQ ID NO: 2 - DNA, 17 bp, synthetic construct
  source 1..17: mol_type = other DNA
cttctactgctcacagg

SEQ ID NO: 3 - DNA, 17 bp, synthetic construct
  source 1..17: mol_type = other DNA
cgggtgcttgccatctt

SEQ ID NO: 4 - DNA, 17 bp, synthetic construct
  source 1..17: mol_type = other DNA
tgccattctgaagccgg

SEQ ID NO: 5 - DNA, 17 bp, Homo sapiens
  source 1..17: mol_type = genomic DNA
cgggtgcttgccatctt

SEQ ID NO: 6 - DNA, 17 bp, Homo sapiens
  source 1..17: mol_type = genomic DNA
gtgcttgccatcttcat

SEQ ID NO: 7 - DNA, 17 bp, Homo sapiens
  source 1..17: mol_type = genomic DNA
cttgccatcttcatctg

SEQ ID NO: 8 - DNA, 17 bp, Homo sapiens
  source 1..17: mol_type = genomic DNA
ccatcttcatctgcagc

SEQ ID NO: 9 - DNA, 17 bp, Homo sapiens
  source 1..17: mol_type = genomic DNA
cagcgggtgcttgccat

SEQ ID NO: 10 - DNA, 17 bp, Homo sapiens
  source 1..17: mol_type = genomic DNA
ttctgccattctgaagc

SEQ ID NO: 11 - DNA, 17 bp, Homo sapiens
  source 1..17: mol_type = genomic DNA
tgccattctgaagccgg

SEQ ID NO: 12 - DNA, 17 bp, Homo sapiens
  source 1..17: mol_type = genomic DNA
ccattctgaagccgggt

SEQ ID NO: 13 - DNA, 17 bp, Homo sapiens
  source 1..17: mol_type = genomic DNA
atcatcttctgccattc

SEQ ID NO: 14 - DNA, 17 bp, Homo sapiens
  source 1..17: mol_type = genomic DNA
atcttctgccattctga

SEQ ID NO: 15 - DNA, 17 bp, Mus musculus
  source 1..17: mol_type = genomic DNA
agtggtaccgtctgcta

SEQ ID NO: 16 - DNA, 17 bp, Mus musculus
  source 1..17: mol_type = genomic DNA
cttctactgctcacagg

SEQ ID NO: 17 - protein, 4 aa, synthetic construct
AEEA

SEQ ID NO: 18 - protein, 5 aa, synthetic construct
HHHHH